(19) United States
(12) Patent Application Publication
(10) Pub. No.: US 2011/0301565 A1
(43) Pub. Date: Dec. 8, 2011

(54) MEDICAL BALLOONS HAVING A SHEATH DESIGNED TO FACILITATE RELEASE OF THERAPEUTIC AGENT

(75) Inventor: Jan WEBER, Maastricht (NL)

(73) Assignee: BOSTON SCIENTIFIC SCIMED, INC., Maple Grove, MN (US)

(21) Appl. No.: 13/094,066

(22) Filed: Apr. 26, 2011

Related U.S. Application Data
(60) Provisional application No. 61/352,117, filed on Jun. 7, 2010.

(51) Int. Cl. A61M 25/10

(52) U.S. Cl. 604/500; 604/103.02

(57) ABSTRACT

Medical devices that comprise an elongate balloon and a sheath positioned around the balloon. The sheath is designed to facilitate the delivery of therapeutic agent. In one embodiment, the sheath has a non-circular shape (e.g., a square shape or polygonal shape). In some cases, the sheath has reservoirs at the corners with a therapeutic agent contained in the reservoirs. In another embodiment, the sheath has an area that undergoes shear strain when the balloon is expanded. The shear strain in the sheath facilitates the release of therapeutic agent. In another embodiment, the sheath has a chamber for containing a therapeutic agent. When the balloon expands, the chamber becomes compressed and causes the therapeutic agent to flow out of the chamber.

### CROSS REFERENCE TO RELATED APPLICATION

The present application claims priority to U.S. provisional application Ser. No. 61/352,117 filed Jun. 7, 2010, the disclosure of which is incorporated herein by reference in its entirety.

### TECHNICAL FIELD

The present invention relates to medical devices, such as balloon catheters, for the delivery of therapeutic agents to body tissue.

### BACKGROUND

Drugs are often delivered directly to target sites of diseased tissue in various contemporary medical procedures. This targeted delivery has proven to be an advantageous approach for treating numerous medical conditions. Using this targeted delivery approach, a controlled dose of the drug may be delivered directly to the target tissue while avoiding or minimizing exposure of other parts of the body to the drug. Also, greater amounts of drug may be delivered to the afflicted parts of the body. In one approach to localized drug delivery, catheter-based, minimally invasive medical procedures are used for deploying devices such as stents, grafts, balloon catheters, and other intravascular devices.

One of the problems that can be encountered with such techniques is inadequate drug release or inadequate control of drug release when the balloon is deployed. For example, in conventional drug-coated balloons, much of the drug can be lost due to washing away by the flow of blood as the balloon is being delivered to the target site. Therefore, there is a need for improved methods for delivering drugs to a target site using a medical balloon.

### SUMMARY

The present disclosure relates to medical devices that use a balloon for delivery of therapeutic agents and methods of medical treatment using such devices. The medical device uses a specially designed sheath around the balloon for improving the release of therapeutic agents.

In one embodiment, the medical device comprises: an elongate balloon; and an expandable sheath positioned around the balloon, the sheath having a non-circular shape on a transverse cross-section; wherein the sheath has a reservoir for containing a therapeutic agent.

In another embodiment, the method of medical treatment comprises: inserting into a patient's body, a medical device comprising: (a) an elongate balloon; (b) an expandable sheath positioned around the balloon, the sheath having a non-circular shape on a transverse cross-section, wherein the sheath has a reservoir; and (c) a therapeutic agent contained in the reservoir; and then expanding the balloon.

In another embodiment, the medical device comprises: an elongate balloon; and an expandable sheath positioned around the balloon, the sheath having an area that undergoes shear strain when the balloon is expanded.

In another embodiment, the method of medical treatment comprises: inserting into a patient's body, a medical device comprising: (a) an elongate balloon; (b) an expandable sheath positioned around the balloon; and (c) a therapeutic agent carried by the sheath; and then expanding the balloon, wherein expanding the balloon causes shear strain in an area of the sheath, and wherein the shear strain in the sheath promotes the release of the therapeutic agent.

In another embodiment, the medical device comprises: an elongate balloon; an expandable sheath positioned around the balloon; a chamber for containing a therapeutic agent, the chamber being located within the sheath; and an opening on the outer surface of the sheath, the opening being in fluid communication with the chamber.

In another embodiment, the method of medical treatment comprises: inserting into a patient's body, a medical device comprising: (a) an elongate balloon; (b) an expandable sheath positioned around the balloon; (c) a chamber for containing a therapeutic agent, the chamber being located within the sheath; (d) an opening on the outer surface of the sheath, the opening being in fluid communication with the chamber; and (e) a therapeutic agent contained in the chamber; and then expanding the balloon, wherein expanding the balloon causes the chamber to be compressed.

### BRIEF DESCRIPTION OF THE DRAWINGS

FIGS. 1A and 1B show a medical device according to one particular embodiment.

FIG. 1A shows a perspective view of the medical device. FIG. 1B shows a transverse cross-section view of the balloon and sheath.

FIGS. 2A and 2B show close-up cross-section views of a corner of the sheath shown in FIGS. 1A and 1B. FIG. 2A shows the corner before the balloon is inflated. FIG. 2B shows the corner as the balloon is being inflated.

FIGS. 3A-3C (transverse cross-section views) show some of the various different shapes that the sheath may have.

FIG. 4A shows a rounded corner for a sheath. FIG. 4B shows a sharp corner for a sheath.

FIG. 5 (transverse cross-section view) shows a sheath having a lobulated shape.

FIGS. 6A and 6B show a portion of a sheath undergoing shear strain.

FIG. 7 shows a portion of a prior art sheath undergoing expansion.

FIGS. 8A-8D show a medical device according to another embodiment. FIG. 8A shows a perspective view of the medical device. FIG. 8B shows a side view of the medical device. FIG. 8C shows a cross-section side view of a portion of the sheath. FIG. 8D shows a side view of the medical device after inflation of the balloon.

FIG. 9 shows a cross-section side view of a portion of a sheath in another embodiment of the medical device.

FIG. 10 shows a perspective view of a sheath having multiple, circumferentially oriented wires embedded in the wall of the sheath.

FIGS. 11A-11D show transverse cross-sections of the sheath shown in FIG. 10 at different longitudinal locations along the sheath.

FIGS. 12A-12C show a sheath having non-uniform wall thickness. FIG. 12A shows a perspective view of the sheath. FIGS. 12B and 12C show transverse cross-sections of the sheath before and after expansion.

FIGS. 13A-13D show transverse cross-sections of the sheath shown in FIG. 12A at different longitudinal locations along the sheath.

FIGS. 14A-14C show a medical device according to another embodiment. FIG. 14A shows a perspective view of the medical device. FIG. 14B shows a transverse cross-section view of a portion of the sheath prior to inflation of the balloon. FIG. 14C shows a transverse cross-section view of the sheath after inflation of the balloon.

### DETAILED DESCRIPTION

Disclosed herein are medical devices that comprise an elongate balloon and an expandable sheath positioned around the balloon. The balloon can be any balloon suitable for medical use, including angioplasty balloons, stent deployment balloons, or other balloons for treating arterial blood vessels. The balloon may have varying degrees of compliance, depending upon the particular application. For example, the balloon may be a compliant, non-compliant, or a semi-compliant balloon. As used herein, a “non-compliant balloon” means a balloon whose diameter increases by no more than 10 percent of the rated nominal diameter as the internal pressure in the balloon is increased above the nominal inflation pressure. As used herein, a “semi-compliant balloon” means a balloon whose diameter increases by 10-20 percent of the rated nominal diameter as the internal pressure in the balloon is increased above the nominal inflation pressure. As used herein, a “compliant balloon” means a balloon whose diameter increases by more than 20 percent of the rated nominal diameter as the internal pressure in the balloon is increased above the nominal inflation pressure. For coronary artery balloons, nominal diameters may range from 1.5-7.0 millimeters (mm), and in the most typical cases, from 2.0-4.0 mm. However, other nominal balloon diameters are also possible, depending upon the intended target site and/or the particular application.

The expandable sheath is designed to facilitate the delivery of therapeutic agent. The sheath may be elastic (i.e., returning substantially to its original shape after the expanding force is removed) or inelastic. The expandable sheath may comprise various types of deformable materials suitable for use in medical devices for insertion into the body, including elastomeric materials. Examples of elastomeric materials include silicone (such as silicone elastomers), fluoropolymer elastomers, or thermoplastic elastomers (such as thermoplastic polyurethanes, thermoplastic polyesters, and thermoplastic polyamides such as polyether block amide (e.g., PEBAX®)). The thickness of the sheath will vary depending upon the particular application. In some cases, the sheath has a thickness of 10-200 micrometers (μm). The sheath may cover the entire balloon or only a portion of the balloon.

In one embodiment, the sheath has a non-circular shape on a transverse cross-section of the sheath (i.e., a cross-section on a plane that is orthogonal to the longitudinal axis of the sheath). The sheath has one or more reservoirs for containing a therapeutic agent. The reservoirs can be associated with the sheath in any suitable manner, including being located on the sheath, within the sheath, or formed by the sheath (e.g., in folds created by the sheath).

The reservoirs can have any suitable configuration for containing a therapeutic agent. For example, the reservoirs can be pockets, grooves, wells, pits, pores, channels, trenches, or other types of voids in the sheath. The reservoirs can be created in the sheath by any suitable method, including as part of the casting process in making the sheath or using various excavation techniques known in the art, such as techniques for direct-write etching using energetic beams (e.g., laser, ion, or electron), micromachining, microdrilling, or lithographic processes. The reservoirs can also be created by forming folds using the sheath. Another way to make the reservoirs is by inserting removable templates during a casting process used to make the sheath. For example, metal or Teflon® wires can be inserted as a template and be pulled out of the formed sheath after the casting process. In some cases, the medical device is provided with a therapeutic agent contained in the reservoirs. The therapeutic agent can be provided at various time points in the manufacture or use of the medical device. For example, the therapeutic agent may be provided during manufacture of the medical device, or alternatively, the therapeutic agent is placed in the reservoirs at the point of use (e.g., in the operating room prior to insertion of the balloon into a patient).

Inflation of the balloon causes the sheath to expand at least in an outward radial direction. The sheath is designed such that expansion of the sheath causes the volume of the reservoirs to shrink, thereby facilitating the release of the therapeutic agent from the reservoirs. In some cases, the volume of the reservoir shrinks to 75 percent or less of the original volume when the balloon is fully expanded; and in some cases, 50 percent or less of the original volume.

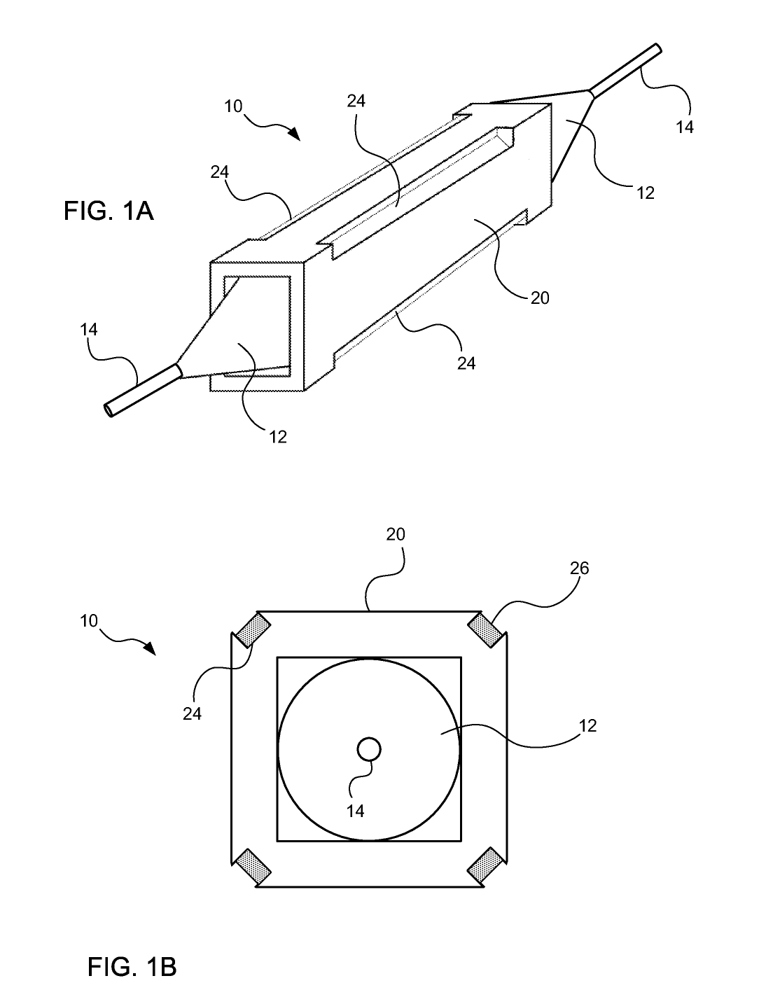

FIGS. 1A and 1B show a medical device 10 according to one particular embodiment. Medical device 10 comprises a non-compliant balloon 12 mounted on a catheter 14. Covering over balloon 12 is a square-shaped, expandable sheath 20, which is made of an elastomeric material such that sheath 20 stretches out as balloon 12 is inflated. Being square-shaped, sheath 20 has four corners where reservoirs 24 are located. Reservoirs 24 contain a therapeutic agent 26.

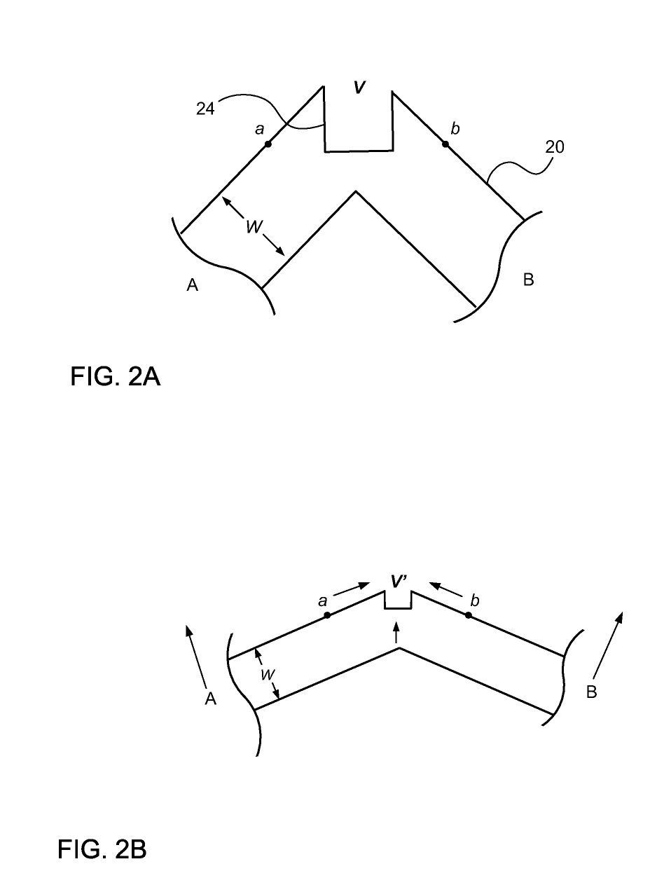

In operation, the medical device 10 is inserted into a patient's body with the balloon 12 in an uninflated state. At the target site in the body (e.g., within a blood vessel, such as an artery), balloon 12 is inflated, causing the expulsion of therapeutic agent 26 out of reservoirs 24. The mechanism by which this occurs is shown in FIGS. 2A and 2B, which show close-up cross-section views of one corner of sheath 20 (therapeutic agent 26 and balloon 12 not shown). FIG. 2A shows the corner prior to inflation of the balloon, with sheath 20 in a square-shaped configuration. Prior to inflation of the balloon, the reservoir 24 at the corner has a volume V. FIG. 2B shows the corner as the balloon is inflated. To facilitate explanation, two segments of sheath 20 on each side of the corner are labeled as segments A and B and two points “a” and “b” are labeled on segments A and B, respectively.

As the balloon is inflated, the segments A and B of sheath 20 expand outward (as shown by the arrows), causing a hinge-like flexion at the corner. This hinge-like flexion at the corner causes the two points “a” and “b” to move toward each other, resulting in the lateral walls of reservoir 24 moving closer to each other. Also, as the diameter of the balloon increases, the sheath 20 is stretched, causing the width W of the sheath 20 to get thinner and the depth of reservoir 24 to get shallower. Together, these movements change the configuration of reservoir 24 such that the volume of reservoir 24 shrinks from volume V to volume V′. This shrinkage in volume facilitates the expulsion of the therapeutic agent out of reservoir 24.

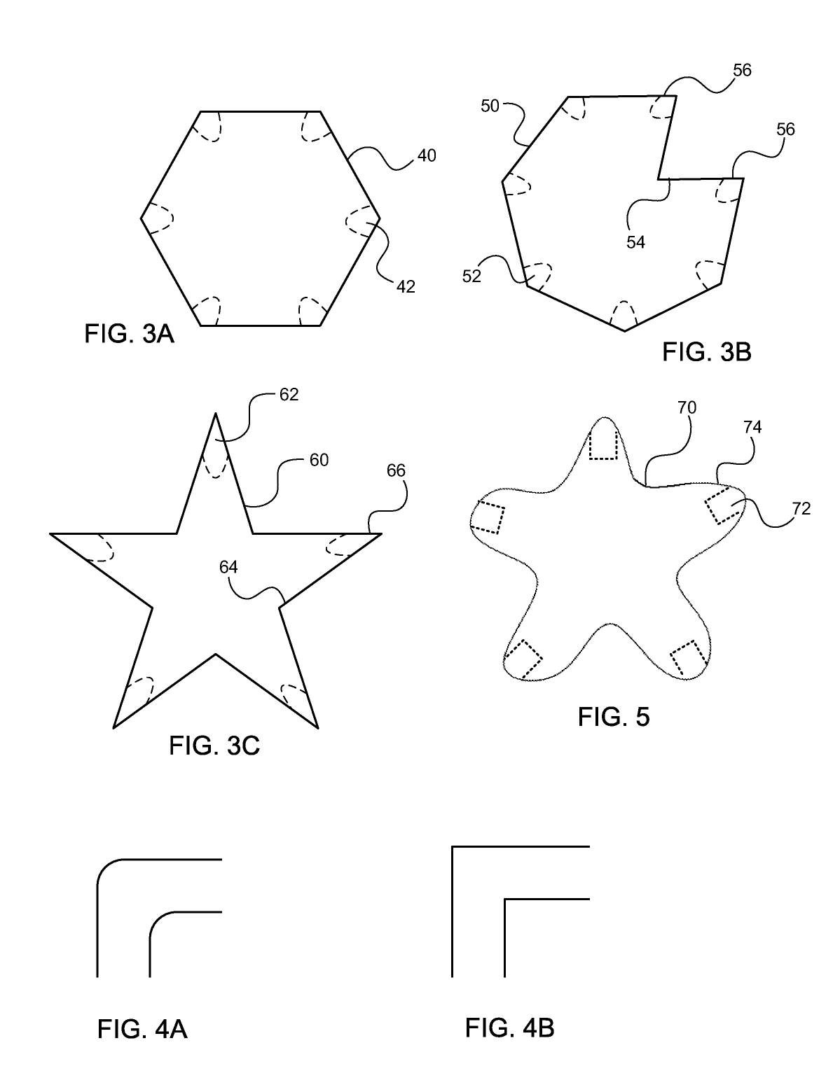

The sheath may have any suitable non-circular shape on its transverse cross-section. In some embodiments, the sheath has a polygonal shape. Examples of polygonal shapes include triangles, squares, pentagons, hexagons, octagons, etc. A polygonal-shaped sheath will have one or more corners. For example, FIG. 3A shows a sheath 40 having a hexagonal shape with reservoirs 42 located at the corners of the sheath 40. Having more corners can be useful in providing a more uniform distribution of the therapeutic agent. The polygonal shapes may have concave as well as convex corners. For example, FIG. 3B shows a sheath 50 having a concave corner 54 and two adjacent convex corners 56, with the reservoirs 52 being located at the convex corners of the sheath 50. In another example, FIG. 3C shows a sheath 60 in a star-shaped configuration with alternating convex corners 66 and concave corners 64, with reservoirs 62 located at the convex corners 66. In a sheath having corners, the corners may be sharp or rounded. For example, FIG. 4A shows a rounded corner, and FIG. 4B shows a sharp corner (reservoirs not shown).

The sheath is not necessarily polygonal in shape and/or does not necessarily have corners. The sheath can have other suitable shapes in which two points on the sheath, such as the points “a” and “b” in FIG. 2B described above, move toward each other as the sheath expands, thereby reducing the volume of the reservoirs. For example, FIG. 5 shows a sheath 70 having lobes 74, with reservoirs 72 located at the lobes 74.

The sheath does not necessarily maintain the same non-circular shape along the entire length of the sheath, so long as least one transverse cross-section of the sheath has a non-circular shape. For example, the non-circular shape does not have to be continuous along the entire length of the sheath. For example, some sections of the sheath may have corners, while other sections of the sheath do not have corners (e.g., having a circular shape). In another example, in a sheath having corners, the corners of the sheath do not necessarily have to follow a straight line in an axial direction. For example, the corners of the sheath may follow a helical direction (e.g., in a twisted configuration). This configuration may be useful in providing a more uniform distribution of therapeutic agent.

To help retain the therapeutic agent within the reservoir during delivery of the balloon to the target site, there may be a barrier coating over the reservoir that degrades or dissolves upon insertion of the balloon in the patient. For example, the barrier coating may comprise a biodegradable or bioresorbable material, such as low-molecular weight carbohydrates (e.g., saccharides or sugars) or biodegradable polymers.

In another embodiment, a medical device of the present disclosure includes an elongate balloon and a sheath having one or more areas that undergo shear strain during expansion of the balloon. A therapeutic agent is carried by the sheath. Deformation of the sheath in the area undergoing shear strain causes the release of the therapeutic agent off the sheath.

The therapeutic agent can be carried by the sheath in any suitable manner. For example, the therapeutic agent may be applied as a coating on the sheath or may be disposed in reservoirs associated with the sheath in a similar manner as explained above. Where the therapeutic agent is disposed in reservoirs, the shear strain causes the reservoirs to shrink in volume to facilitate the release of the therapeutic agent. In some cases, the volume of the reservoir shrinks to 75 percent or less of the original volume when the balloon is fully expanded; and in some cases, 50 percent or less of the original volume.

The therapeutic agent may be provided at various time points in the manufacture or use of the medical device. For example, the therapeutic agent may be provided during manufacture of the medical device, or alternatively, the therapeutic agent may be applied to the sheath at the point of use (e.g., in the operating room prior to insertion of the balloon into a patient).

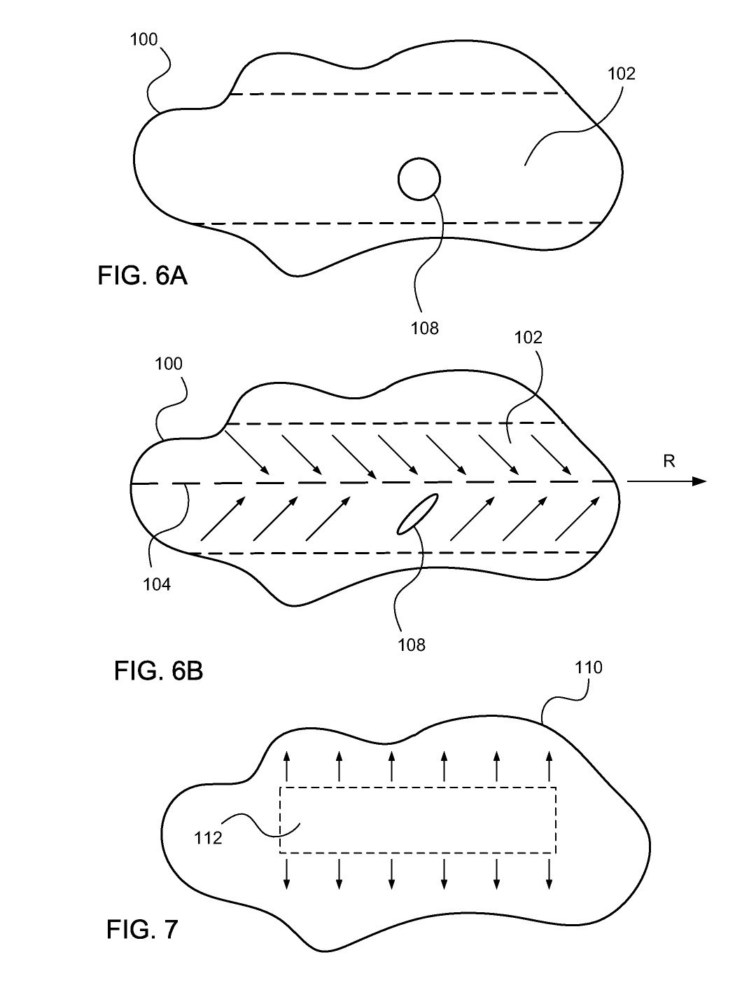

Shear strain is introduced into the sheath by non-uniform stretching of one or more areas of the sheath during expansion. The area undergoing shear strain comprises a portion (e.g., along a line, path, or point on the sheath) that moves in a direction that is not a direction that the portion would otherwise take if the sheath was stretching outward in a uniform radial and/or axial direction during expansion of the sheath. FIGS. 6A and 6B show one example of how shear strain can be introduced into a sheath. FIG. 6A shows a top view of a portion of a sheath 100 having an area 102 that undergoes shear strain when the balloon is inflated. Located within this area 102 is a reservoir 108 having a round shape, where reservoir 108 contains a therapeutic agent.

As seen in FIG. 6B, upon inflation of the balloon, sheath 100 expands and a portion of sheath 100 along line 104 moves in the direction of arrow R. This causes area 102 to undergo shear strain, as depicted by the arrows in area 102 pointing toward line 104. This shear strain deforms the shape of reservoir 108 such that the volume of reservoir 108 shrinks, thereby facilitating the expulsion of therapeutic agent out of reservoir 108. In contrast, FIG. 7 shows a portion of a conventional, prior art sheath 110 that expands outward radially in a uniform manner (in the direction of the arrows) as the balloon is inflated. In this situation, the area 112 that is being stretched does not undergo shear strain.

The shear strain area may have any suitable shape or geometry, and may be oriented or moved along a path in various directions. For example, the shear strain area may be oriented in a direction or moved along a path that is axial, circumferential, or helical with respect to the sheath. The shear strain may be introduced into the sheath by a variety of different mechanisms. In some cases, one or more connecting members are joined to the sheath for the purpose of creating shear strain in the sheath as the sheath expands. The connecting members are configured such that, as the balloon is expanded, the connecting members pull or push the portions of the sheath to which the connecting members are joined. The connecting members may be less compliant than the sheath (e.g., a sheath made of a compliant polymeric material may have metal wires as connecting members). The connecting members can be incorporated into the sheath using any suitable manufacturing process. For example, the connecting members can be integrated into the sheath during a casting process for making the sheath.

One part of the connecting member is joined to the sheath, and another part of the connecting member is joined to another part of the sheath or another part of the medical device (such as a catheter or balloon). For example, one end of the connecting member may be joined to the sheath and the other end of the connecting member is joined to a part of the medical device that is distal to the sheath (e.g., distal end of the balloon), or joined to a part of the medical device that is proximal to the sheath (e.g., proximal end of the balloon). In some cases, with one part of the connecting member joined to the sheath, another part of the connecting member is joined to a portion of the medical device that is fixed, i.e., does not move relative to the balloon during inflation of the balloon (e.g., a catheter or the distal/proximal ends of the balloon). The connecting members may be wires, hooks, fibers, mesh, or any other structure or material that can connect one part of the sheath to another part of the sheath or another part of the medical device.

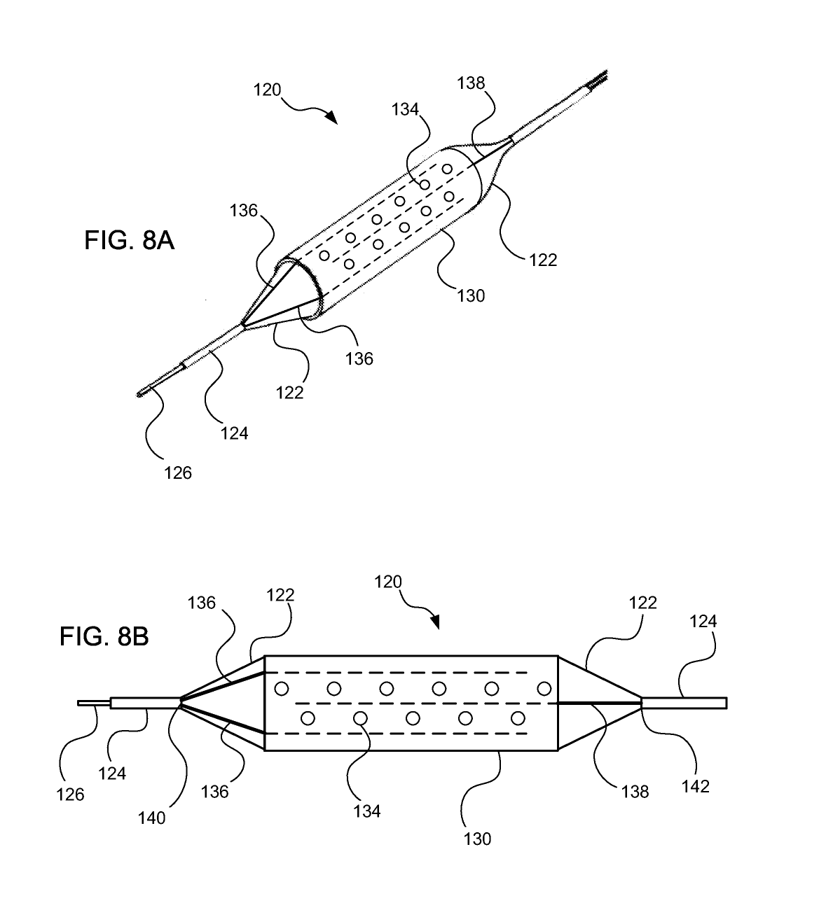

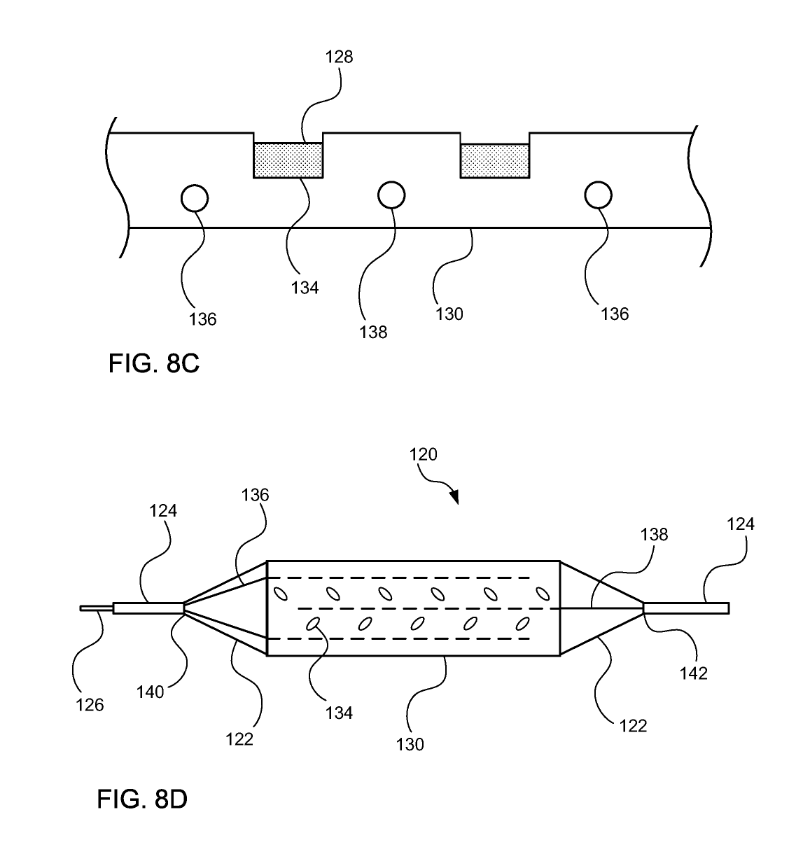

FIGS. 8A-8D show a medical device 120 according to one particular embodiment. As shown here, medical device 120 comprises a non-compliant balloon 122 mounted on a catheter 124 and guidewire 126. An expandable sheath 130 made of an elastomeric material covers balloon 122. Sheath 130 has round-shaped reservoirs 134 that contain a therapeutic agent 128 (see FIG. 8C). Embedded in sheath 130 are wires 136 and 138, which are alternately connected to the distal end 140 or the proximal end 142 of balloon 122. FIG. 8C is a cross-section side view of a portion of sheath 130 showing wires 136 and 138 that are embedded within sheath 130.

In operation, the medical device 120 is inserted into a patient's body with the balloon 122 in an uninflated state. At a target site in the body (e.g., within a blood vessel, such as an artery), balloon 122 is inflated, causing the expulsion of therapeutic agent 128 out of reservoirs 134. The mechanism by which this occurs is shown in FIG. 8D, which shows medical device 120 as balloon 122 is being inflated. Inflation of balloon 122 causes sheath 130 to expand radially outward. However, this outward radial expansion of sheath 130 is not uniform because those portions of sheath 130 along wires 136 and 138 are pulled in opposite, but substantially parallel directions that are offset from each other as sheath 130 expands outward. The portions of sheath 130 along embedded wires 136 are pulled toward the distal end 140 of the balloon 122, and the portions of sheath 130 having embedded wires 138 are pulled toward the proximal end 142 of the balloon 122. This differential movement between these different portions of sheath 130 creates areas of shear strain in sheath 130. The shear strain deforms the shape of each reservoir 134 into a thinner, elliptical shape such that the volume of reservoir 134 shrinks, thereby facilitating the expulsion of therapeutic agent 128 out of reservoir 134.

Sheath 130 having wires 136 and 138 embedded therein can be made using any suitable process. For example, one way to make sheath 130 is to place wires 136 and 138 on a mandrel, and then overspray the wires with polyurethane. In another example, sheath 130 can be made by extrusion of the sheath material with wires 136 and 138.

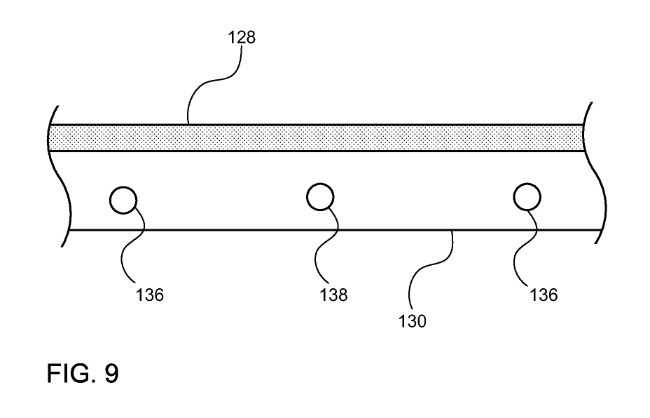

In some embodiments, instead of the therapeutic agent being contained in reservoirs, the therapeutic agent may be provided as a coating on the sheath. For example, FIG. 9 shows a cross-section side view of the portion of sheath 130 shown in FIGS. 8A-8D, except that therapeutic agent 128 is provided as a coating instead of being contained in reservoirs. In this embodiment, the shear strain in sheath 130 can promote the breakage and/or detachment of the coating of therapeutic agent 128, thus facilitating release of the therapeutic agent 128. This configuration can be advantageous because the shear strain areas can be designed to promote the breakage and/or detachment of the coating in a more controlled manner. For example, the shear strain areas can be designed to promote the breakage of the coating into particles or fragments of more uniform size or into sizes that are more therapeutically effective.

In some cases, one or more stretch limiting elements are joined to the sheath for the purpose of creating shear strain in the sheath as the sheath expands. One end of the stretch limiting element is joined to one part of the sheath and the other end of the stretch limiting element is joined to another part of the sheath. The stretch limiting elements may be less compliant than the sheath (e.g., a sheath made of a compliant polymeric material may have metal wires as stretch limiting elements). The stretch limiting elements may be wires, hooks, fibers, mesh, or any other structure or material that can connect one part of the sheath to another part of the sheath. The stretch limiting elements can be joined to the sheath in any suitable configuration to cause non-uniform stretching of the sheath as the sheath expands.

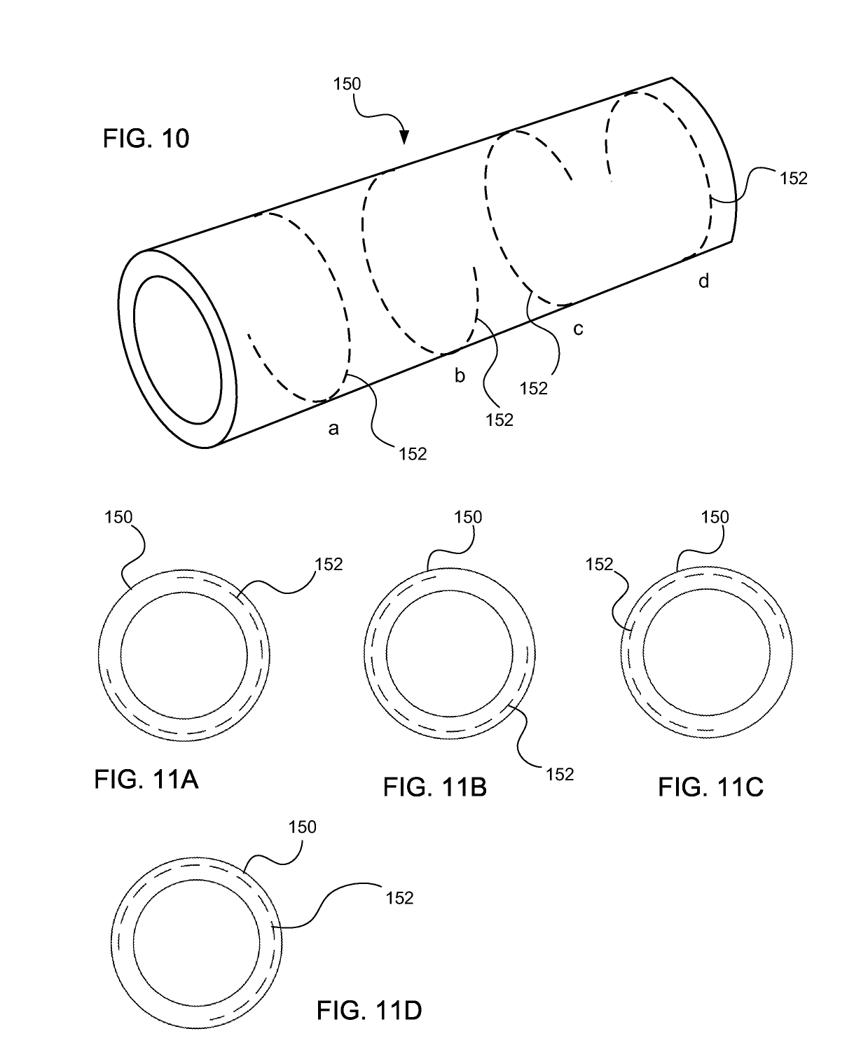

FIG. 10 shows a sheath 150 according to one particular embodiment. Sheath 150 has multiple fibers 152 embedded within its wall. Fibers 152 are spaced apart longitudinally along sheath 150 and travel in a circumferential path around sheath 150. The fibers 152 only partially encircle the sheath. Furthermore, the circumferential arc of fibers 152 around sheath 150 occupy different angular sections of sheath 150. This is shown more clearly in FIGS. 11A-11D, which show transverse cross-sections of sheath 150 at sections a, b, c, and d in FIG. 10. FIG. 11A shows a transverse cross-section of sheath 150 at section a with fiber 152 embedded therein. The path of fiber 152 is a 270° arc around sheath 150. Going from FIG. 11A to 11D, the circumferential arc of fibers 152 are located at different angular sections of sheath 150. In operation, when sheath 150 is expanded, the differential expansion of sheath 150 along the line where fibers 152 are embedded causes shear strain in sheath 150.

This is because the fibers 152 act as stretch limiting elements that limit the stretch of the sheath 150 in the areas where they are present. Thus, in the example sections shown in FIGS. 11A-11D, the sheath 150 will stretch more in the 90° area where the stretch limiting element is absent than in the 270° area where the stretch limiting element is present. This induces differential expansion and shear strain in the sheath 150.

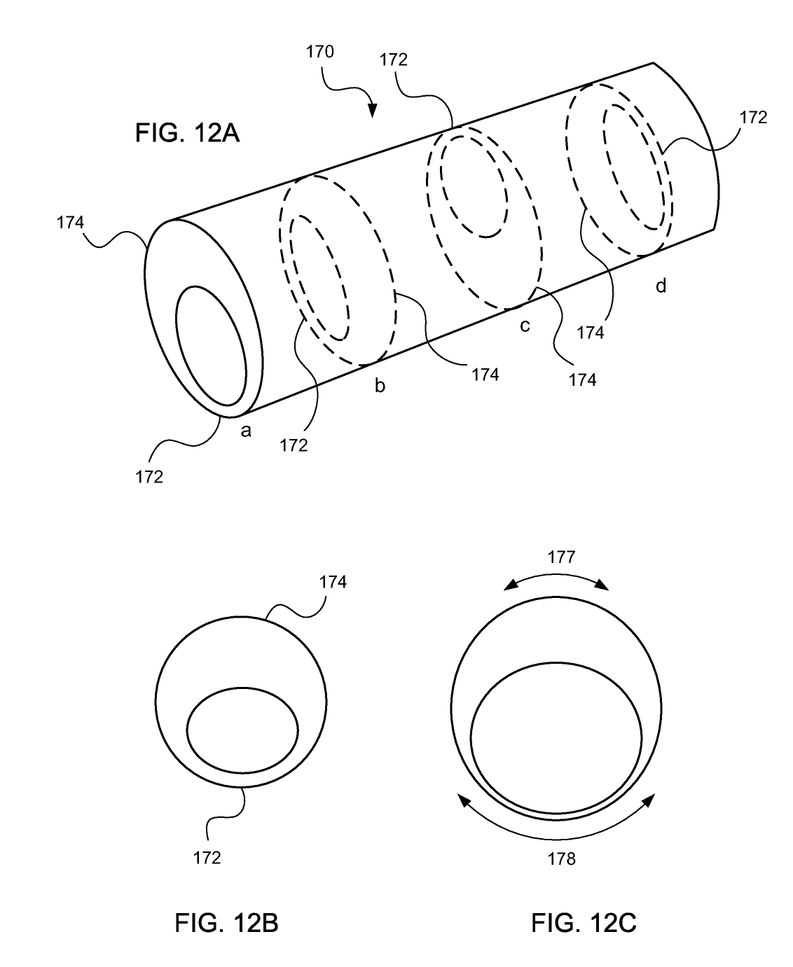

Another way of designing a sheath to have area(s) that undergo shear strain upon expansion is to make the sheath with non-uniform wall thickness. The differences in the wall thickness of the sheath can cause non-uniform stretching of the sheath during expansion, thereby introducing shear strain into the sheath. In some cases, the wall of the sheath may have a non-uniform thickness as measured along a circumferential path on the sheath, or a longitudinal path on the sheath, or both. For example, FIG. 12A shows a sheath 170 according to one particular embodiment. Sheath 170 has non-uniform thickness with thinner portions 172 and thicker portions 174. FIGS. 12B and 12C show transverse cross-sections of sheath 170 and demonstrate the relationship between the thinner portion 172 and thicker portion 174. FIGS. 12B and 12C also demonstrate how thinner portion 172 expands differently compared to thicker portion 174. FIG. 12B shows sheath 170 in an unexpanded state and FIG. 12C shows how sheath 170 expands with thinner portion 172 stretching to a greater extent than thicker portion 174 (see arrow 178 compared to arrow 177). In some cases, the thicker portions 174 are between 10-100 μm thicker than thinner portions 172. In some cases, the thicker portions 174 are at least 10 μm thicker; and in some cases, at least 20 μm thicker; and in some cases, at least 50 μm thicker than thinner portions 172.

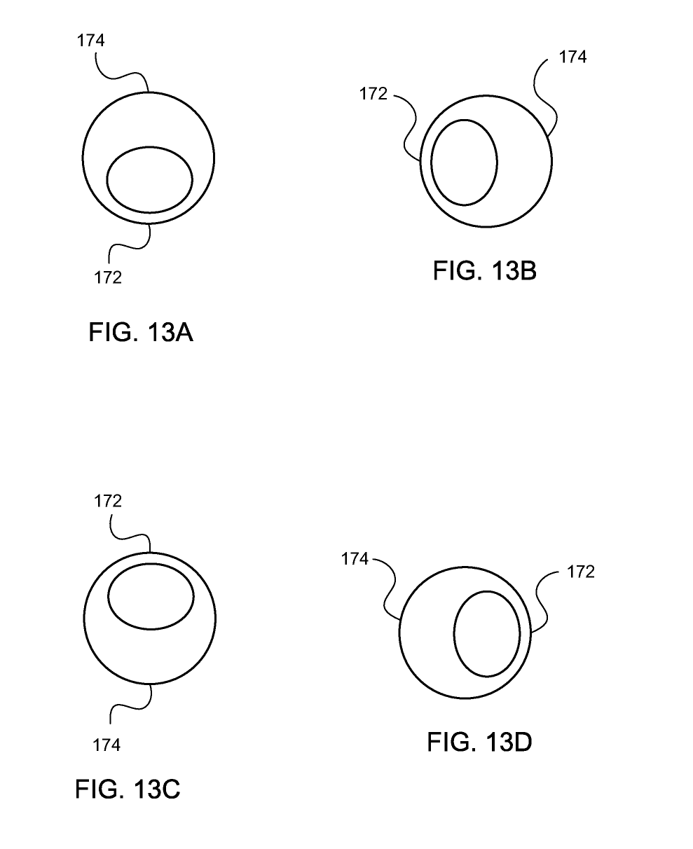

The pattern of the non-uniformities in the sheath thickness can be designed to promote non-uniform stretching of the sheath. In the example of sheath 170, the thinner portions 172 and thicker portions 174 take a spiral path along the sheath. The effect of this is shown more clearly in FIGS. 13A-13D, which show transverse cross-sections of sheath 170 at sections a, b, c, and d shown in FIG. 12A. In each of the sections shown in FIGS. 13A-13D, the thinner portions 172 and thicker portions 174 have different circumferential locations. Thus, sheath 170 has a non-uniform thickness in a circumferential direction as well as a longitudinal direction along sheath 170. In operation, when sheath 170 is expanded, the differential expansion at the thinner portions 172 relative to the thicker portions 174 causes shear strain in sheath 170.

Another way of designing a sheath to have area(s) that undergo shear strain upon expansion is to make the sheath with non-uniform compliance. In such embodiments, one or more portions of the sheath are less compliant (or more compliant) compared to other portion(s) of the sheath. The pattern of the non-uniformities in the sheath compliance is designed to promote non-uniform stretching of the sheath, thereby introducing shear strain into the sheath. A sheath having non-uniform compliance can be made in various ways. For example, in a sheath made of a polymeric material, portions of the sheath can be made less compliant by subjecting it to localized UV radiation to crosslink the polymeric material.

In another embodiment, a medical device of the present disclosure includes an elongate balloon and a sheath having one or more internal chambers within the sheath. The internal chambers can have any suitable configuration for containing a therapeutic agent, such as channels, passageways, cavities, or other types of voids. The sheath further comprises openings (such as pores, holes, or slits) at the outer surface of the sheath. The openings are in fluid communication with one or more of the internal chambers. A therapeutic agent is contained in the chamber, which may be provided at various time points in the manufacture or use of the medical device. For example, the therapeutic agent may be provided during manufacture of the medical device, or alternatively, the therapeutic agent may be applied at the point of use (e.g., in the operating room prior to balloon insertion, or even after insertion of the balloon into the patient (e.g., by infusion through a catheter)).

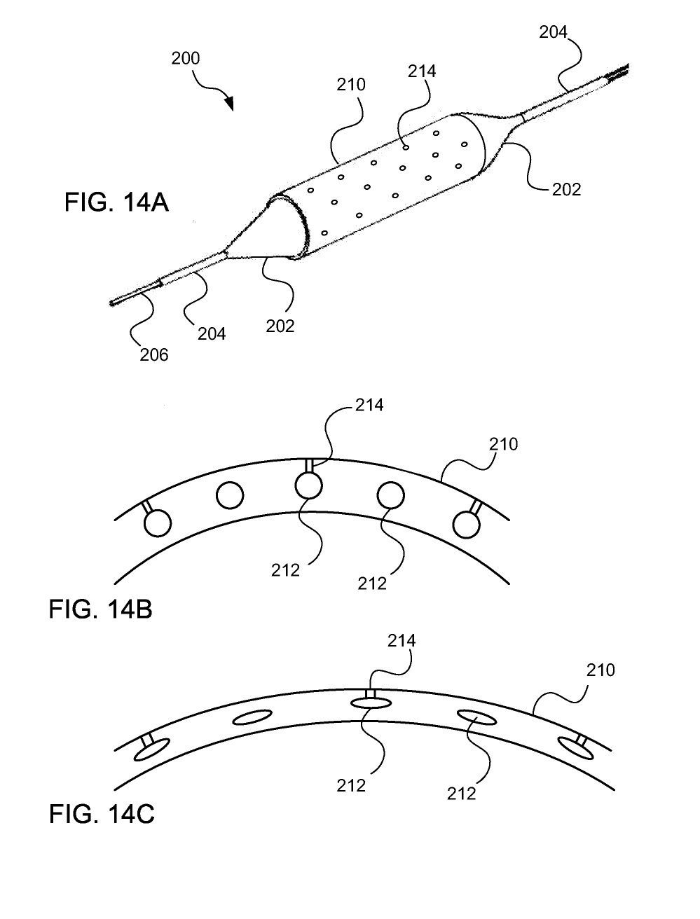

FIGS. 14A-14C show a medical device 200 according to one particular embodiment. As shown here, a medical device 200 comprises a non-compliant balloon 202 mounted on a catheter 204 and guidewire 206. An expandable sheath 210 made of an elastomeric material covers over balloon 202. As seen in FIG. 14B, within sheath 210 are internal chambers 212 that contain a therapeutic agent. In fluid communication with internal chambers 212 are pores 214 that lead to an opening on the surface of sheath 210 so that the therapeutic agent can flow out of internal chambers 212. As shown in FIG. 14C, when balloon 202 (not shown) is inflated, sheath 210 stretches out and becomes thinner. This compresses internal chambers 212 and reduces their volume, thereby causing the therapeutic agent to flow out of internal chambers 212 and become released through pores 214. Thinning of sheath 210 as it stretches out can be facilitated by increasing the compliance of the sheath 210 (e.g., by using an elastomeric material to make the sheath, as described above).

The internal chambers can be made within the sheath using any suitable process. For example, one way to make the internal chambers is to spray a layer of polyurethane on a mandrel. After the polyurethane dries, a series of wires are placed on the polyurethane layer and oversprayed with more polyurethane. After drying, the wires are pulled out or dissolved to create longitudinal channels within a polyurethane sheath.

The therapeutic agent used in the medical devices disclosed herein may be a pharmaceutically acceptable agent (such as a drug), a biomolecule, a small molecule, or cells. Exemplary drugs include anti-proliferative agents such as paclitaxel, sirolimus (rapamycin), tacrolimus, everolimus, biolimus, and zotarolimus. Exemplary biomolecules include peptides, polypeptides and proteins; antibodies; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Exemplary small molecules include hormones, nucleotides, amino acids, sugars, and lipids and compounds have a molecular weight of less than 100 kD. Exemplary cells include stem cells, progenitor cells, endothelial cells, adult cardiomyocytes, bone marrow cells, and smooth muscle cells. Other therapeutic agents that may be used in the present invention include those listed in U.S. Pat. No. 7,572,625 (Davis et al., “Medical devices coated with drug carrier macromolecules”), which is incorporated by reference herein. Any of the therapeutic agents may be combined to the extent such combination is biologically compatible.

The therapeutic agent may be provided in combination with one or more other materials. For example, the therapeutic agent can be blended with additives or excipient materials (e.g., binders, plasticizers, fillers, etc.). The therapeutic agent may be provided in any suitable formulation or dosage form, such as within capsules or as nanoparticles (e.g., albumin-bound paclitaxel, sold as Abraxane® (Astra-Zeneca)).

Medical devices of the present invention may also include a vascular stent mounted on the balloon. The vascular stent may be those known in the art, including stents with or without coatings that elute a therapeutic agent. The stent may also be biostable, bioerodable, or biodegradable. The stent may be a bare stent or may have a drug coating.

The balloons or sheaths of the present disclosure may also be coated with a low-molecular weight carbohydrate, such as mannitol. The carbohydrate may be a separate coating or be blended with the therapeutic agent. The balloons or sheaths of the present disclosure may also be coated with a radiocontrast agent (ionic or non-ionic), such as iopromide, bismuth subcarbonate, bismuth oxychloride, bismuth trioxide, barium sulfate, tungsten, and mixtures thereof. The contrast agent may also be a magnetic contrast agent (e.g., ferromagnetic or paramagnetic) such as iron oxides, dysprosium oxides, or gadolinium oxides. The contrast agent may be a separate coating or be blended with the therapeutic agent. The balloons or sheaths of the present disclosure may also be coated with a water-soluble polymer, such as polyvinylpyrrolidone (PVP). The polymer may be a separate coating or be blended with the therapeutic agent.

The foregoing description and examples have been set forth merely to illustrate the invention and are not intended to be limiting. Each of the disclosed aspects and embodiments of the present invention may be considered individually or in combination with other aspects, embodiments, and variations of the invention. Modifications of the disclosed embodiments incorporating the spirit and substance of the invention may occur to persons skilled in the art and such modifications are within the scope of the present invention.

What is claimed is:

1. A medical device comprising:
an elongate balloon; and
an expandable sheath positioned around the balloon, the sheath having a non-circular shape on a transverse cross-section;
wherein the sheath has a reservoir for containing a therapeutic agent.

2. The medical device of claim 1, wherein the sheath has two separate points on the sheath that move closer to each other when the balloon is expanded, and wherein the reservoir is located between the two points.

3. The medical device of claim 2, wherein the two points are located on opposing edges of the reservoir.

4. The medical device of claim 2, wherein the two points are located on the same transverse plane through the sheath.

5. The medical device of claim 1, wherein the sheath has a corner and the reservoir is located at the corner.

6. The medical device of claim 5, wherein the reservoir extends longitudinally along the corner of the sheath.

7. The medical device of claim 1, wherein the sheath has a polygonal shape with a plurality of corners and a reservoir located at a corner of the sheath.

8. The medical device of claim 1, wherein the sheath has a lobe, and wherein the reservoir is located at the lobe.

9. The medical device of claim 1, wherein expanding the balloon causes the volume of the reservoir to shrink.

10. The medical device of claim 1, further comprising a therapeutic agent contained in the reservoir.

11. A method of medical treatment comprising:
inserting into a patient's body, a medical device comprising:
(a) an elongate balloon;
(b) an expandable sheath positioned around the balloon, the sheath having a non-circular shape on a transverse cross-section, wherein the sheath has a reservoir; and
(c) a therapeutic agent contained in the reservoir;
expanding the balloon.

12. A medical device comprising:
an elongate balloon; and
an expandable sheath positioned around the balloon, the sheath having an area that undergoes shear strain when the balloon is expanded.

13. The medical device of claim 12, wherein the area that undergoes shear strain includes a first portion that moves along a first path when the balloon is expanded.

14. The medical device of claim 13, wherein the area that undergoes shear strain further includes a second portion that moves along a second path when the balloon is expanded, wherein the second path is in a different direction than the first path.

15. The medical device of claim 14, wherein the first path is in an opposite and substantially parallel offset direction from the second path.

16. The medical device of claim 13, further comprising:
a first connecting member joined to the first portion on the sheath;
wherein expansion of the balloon causes the first connecting member to move the first portion along the first path.

17. The medical device of claim 14, further comprising:
a first connecting member joined to the first portion on the sheath; and
a second connecting member joined to the second portion on the sheath;
wherein expansion of the balloon causes the first connecting member to move the first portion along the first path and causes the second connecting member to move the second portion along the second path.

18. The medical device of claim 12, further comprising a therapeutic agent carried by the sheath.

19. The medical device of claim 18, wherein the sheath has a reservoir and the therapeutic agent is contained in the reservoir.
